(19) United States
(12) Patent Application Publication
(10) Pub. No.: US 2009/0312822 A1
(43) Pub. Date: Dec. 17, 2009

(54) Far Infrared Heat Emitting Therapeutic Device

(76) Inventor: Robert Besner, Boca Raton, FL (US)

Correspondence Address: James David Johnson, P.A., 401 East Las Olas Boulevard, Suite 130-290, Fort Lauderdale, FL, 33301, US

(21) Appl. No.: 12/485,807

(22) Filed: Jun. 16, 2009

Related U.S. Application Data
(60) Provisional application No. 61/061,813, filed on Jun. 16, 2008.

(51) Int. Cl. A61F 7/00

(52) U.S. Cl. 607/100

(57) ABSTRACT

A far infrared heat emitting device includes a pad for positioning over or around a body part of a user such as a waist, leg, arm, or back. The user may be a human or an animal such as a pet. The device can include straps to form a belt sized to fit around the waist of a human user. The belt includes a pad, a plurality of far infrared heat emitting elements, a charging inlet, a heating device, and a fastener for securing the belt to the human user. The device may be secured around the waist of the human user to provide relief from lower back pain.

### CROSS-REFERENCE TO RELATED APPLICATION

This application claims the priority from U.S. provisional patent application Ser. No. 61/061,813 filed Jun. 16, 2008. The foregoing application is incorporated in its entirety herein by reference.

### FIELD OF THE INVENTION

The invention relates to devices and methods for providing healing or curative effects for health conditions as well as pain relief and relaxation. More particularly, the invention relates to devices and methods that incorporate a far infrared heating system that is useful for relaxing muscles, relieving pain, and increasing blood circulation.

### BACKGROUND

One of the most common medical complaints is lower back pain. Lower back pain is associated with inflammation of the nerves, muscles, bones, connective tissue, or joints and may result from a number of different causes. Among the many different treatments for lower back pain, heat treatment has been shown to be both effective and easy to administer. See, French et al., (2006) Spine 31:998-1006.

Conventional heat emitting therapeutic devices have utilized heated water in the form of hot water bags and bottles, oil containing bags or bladders that can be heated, and electric blankets. These conventional heating devices heat the body's surface through heat transfer by thermal radiation and heat conduction between the heating device and the skin. Often, the thermal radiation transferred as heat between two surfaces is near infrared radiation or a more narrow spectrum of infrared radiation, and as a result, the wavelength of infrared radiation emitted may not penetrate deeply enough into or beneath the skin to provide any useful curative or pain relief effects. For example, near infrared radiation emitted by conventional heating devices may only penetrate one to two millimeters beneath the skin and fail to provide pain relief effects. In addition, because of the nature of human skin protection our bodies, conventional heating emitting therapeutic devices may only be used for a short period of time before burning of the skin occurs.

### SUMMARY

The invention relates to far infrared (FIR) heat emitting devices that generate heat in the form of FIR radiation that is emitted and transferred both by radiation and conduction to a human's skin surface for conduction into and penetration beneath the skin. Due to the wavelength of FIR heat waves, the FIR waves can penetrate more deeply into and beneath the skin surface to provide curative, pain relieving effects for tissue beneath the surface of the skin.

The FIR heat emitting device can be a therapeutic device that emits FIR heat that can be absorbed by the human body, and particularly, through human skin and sub-dermal tissues, through FIR heat radiation and conduction. The therapeutic device can be a pad that features heat emitting and heat conducting and heat radiating elements. The FIR heat emitting device can include a heating device such as a frequency generating device connected to a power source and including a resonating element. When the frequency generating device is connected to a power source, the electricity causes the resonating element to resonate, which in turn, produces far infrared radiation that can be emitted through the pad.

Far infrared heat emitting elements (e.g., jade discs) installed on an exterior surface of the pad can be exposed to some of the radiating far infrared heat emitted by the resonating element. The FIR heat emitting elements can be made from a material that also exhibits resonance when exposed to a particular frequency of electromagnetic radiation, and in particular, when exposed to frequencies in the far infrared segment of the electromagnetic spectrum. As the FIR heat emitting elements resonate (or vibrate), they emit far infrared heat that can penetrate the user's skin and tissues by conduction (direct contact) to provide pain relief and relaxation. The heating conducting elements can be a plurality of stone or mineral members such as, for example, jade. The heating elements can be arranged in a regular pattern on an inner or outer surface of the pad. The pad can further include a charger to permit connection to a power source.

In some embodiments, the pad may include one or more fasteners to form a belt that can be attached around a body part such as, for example, a waist, arm, leg, or neck. The fasteners can be straps that can include buckle, button, or hook-and-loop fastening means.

The FIR heat emitting belt can conveniently be worn around the waist of a human subject to relieve lower back pain. In preferred embodiments, the belt is shaped somewhat like a conventional weight lifting belt for the purpose of securing around a user's waist and is flexible. The belt may include a bladder that contains a fluid that becomes heated when the belt is being charged (e.g., wherein a 15 minute charge can provide about 4 hours of heat). Far infrared heat can be delivered to a user via jade discs arranged in a regular pattern on or beneath a front surface of the belt such that the jade discs are positioned in contact or near contact with the lower back when the belt is being worn. A belt extension, or an optional elastic strap, can be used to secure the belt to a chair.

Accordingly, the invention features a therapeutic device for positioning over or around a body part of a user. The device can feature a pad that includes a front surface and a rear surface, a plurality of far infrared heat emitting elements affixed to the pad, a heating device, and a power source.

In another aspect, the invention can feature a bladder containing a heat storing liquid.

In another aspect, the invention can feature the pad including a fastener to form a belt for securing the pad around a body part of the user.

In another aspect, the invention can feature the belt being integrated with the pad so that the belt is formed as a single piece.

In another aspect, the invention can feature the belt including a short extension for securing the belt around waists of sizes 36-48 inches.

In another aspect, the invention can feature the belt including a long extension for securing the belt around waists of size 48 inches or greater.

In another aspect, the invention can feature the device being sized so as to be capable of attaching around a chair.

In another aspect, the invention can feature the power source including a power cord for connection to an outlet of an electrical system.

In another aspect, the invention can feature the power source including a battery.

In another aspect, the invention can feature the plurality of far infrared heat emitting elements being constructed from a stone or a mineral.

In another aspect, the invention can feature the plurality of far infrared heat emitting elements being constructed from jade.

In another aspect, the invention can feature the front surface of the pad including a pouch, wherein the pouch features a lining comprising front and rear interior surfaces.

In another aspect, the invention can feature the pad including individual cells sewn into a lining of the pouch.

In another aspect, the invention can feature each of the plurality of far infrared heat emitting elements being positioned within one of the individual cells sewn into the lining of the pouch.

The invention can also feature a far infrared heat emitting device that includes a belt. The belt can feature a plurality of far infrared heat emitting elements, a heating device, a charging inlet, a fastener for securing the belt to a user, and a power source.

In another aspect, the invention can feature the belt being sized to fit around the waist of the user.

The invention also features a method of administering far infrared heat to a body part of a user. The method can include the step of placing a far infrared heat emitting device in contact with a body part of a user. The far infrared heat emitting device can feature a pad that includes a plurality of far infrared heat emitting elements, a heating device, and a power source.

Another method of the invention can include the step of leaving the pad of the device in position in contact with the body part of the user for a predetermined amount of time.

Another method of the invention can include the step of leaving the pad of the device in position in contact with the body part of the user until the user's pain is diminished.

Another method of the invention can include the steps of equipping the pad with a fastener to form a belt, and attaching the belt around the body part of the user.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### BRIEF DESCRIPTION OF THE DRAWINGS

FIG. 1 is a front view of a pad embodiment of the invention.

FIG. 2 is a rear view of the pad embodiment of FIG. 2.

FIG. 3 is a front view of a belt embodiment of the invention.

FIG. 4 is a rear view of the belt embodiment of FIG. 3.

FIG. 5A is a top view of one side of a short belt extension capable of being connected to and used with the belt embodiment of FIG. 3.

FIG. 5B is a top view of a reverse side of the short belt extension of FIG. 5A.

FIG. 6A is a top view of one side of a long belt extension capable of being connected to and used with the belt embodiment of FIG. 3.

FIG. 6B is a top view of a reverse side of the long belt extension of FIG. 6A.

FIG. 7A is a front perspective view of the belt embodiment of FIG. 3 connected to a long belt extension and attached around a chair.

FIG. 7B is a rear perspective view of the belt embodiment of FIG. 3 connected to the long belt extension as shown and attached around a chair.

### DETAILED DESCRIPTION

The invention provides a far infrared (FIR) heat emitting device that can be worn or placed over, under, or around a body part of a user such as, for example, a waist, an arm, a leg, a neck, a torso, a hand, a foot, or a back of a human subject. In another embodiment, the user of the invention described herein can be an animal such as a domestic animal, a pet, a farm animal, or a work animal, e.g., a dog, a cat, a horse, a cow, a pig, or any other animal on which a FIR heat emitting device may be used.

In one exemplary embodiment, the FR heat emitting device 10 can include a pad 12 that is laid or placed over and in contact with or wrapped around a body part of a user. In another exemplary embodiment, the pad 12 can be equipped with one or more fasteners to form a FIR heat emitting device that is a belt 100. The belt 100 can be attached around a body part of the user. The FIR heat emitting device can further include a plurality of far infrared heat emitting elements 14.

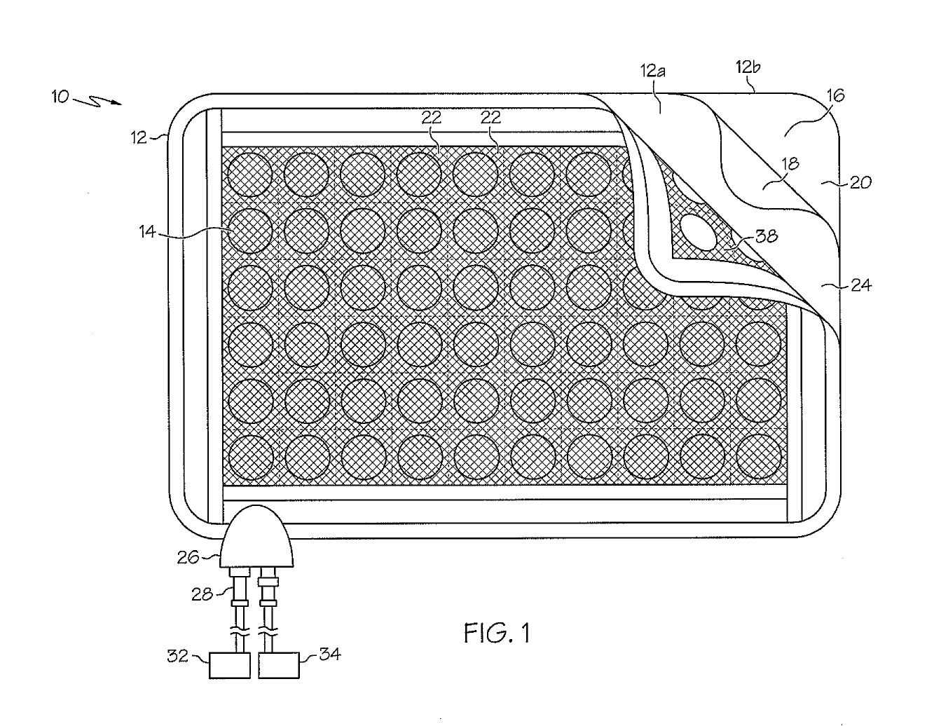

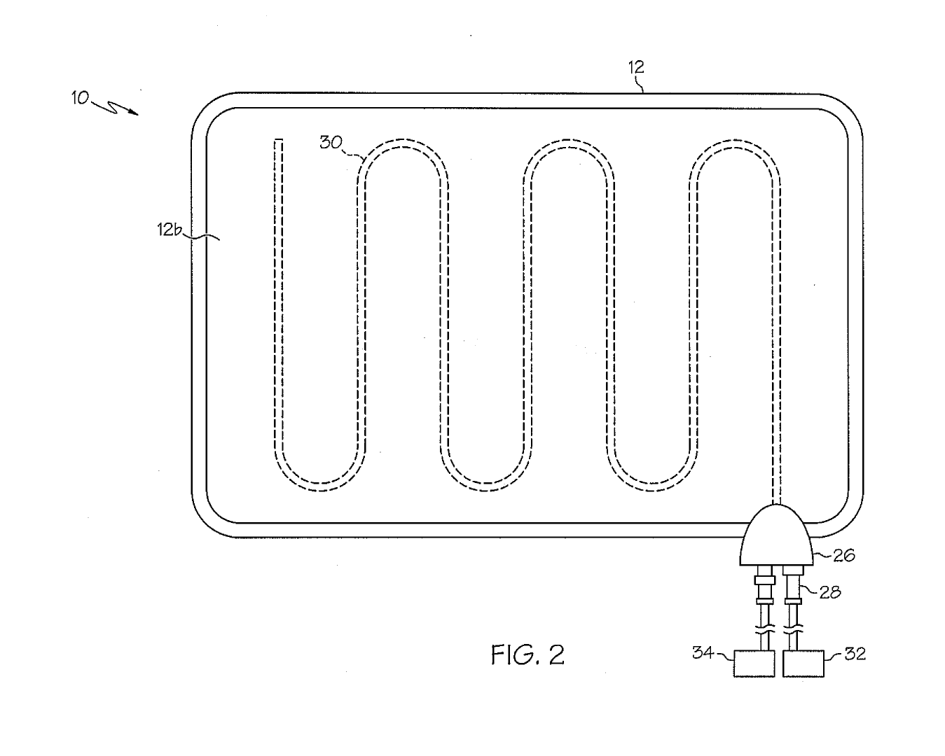

In an exemplary embodiment shown in FIGS. 1 and 2, the pad 12 can be generally flat having a front sheet of material 12a and a rear sheet of material 12b connected together around at least an outside edge of each sheet of material. The front sheet of material 12a and the rear sheet of material 12b may be attached together by stitching. The front and rear sheets of material 12a and 12b may define an empty space between them so as to form a pouch 16. The pouch 16 can be defined by an interior surface 18 of the front sheet of material 12a and an interior surface 20 of the rear sheet of material 12b.

The device 10 can include a heating device 30. The heating device 30 can be a frequency generating device 30. The frequency generating device 30 can be installed in or on the pad 12 and can be capable of generating a frequency that causes the plurality of FIR heat emitting elements 14 to resonate or vibrate. As the plurality of FIR heat emitting elements 14 vibrate, the elements generate heat in the form of far infrared heat radiation that can be emitted by the elements onto the skin of the user.

In an exemplary embodiment, the frequency generating device can feature a carbon-based, polygraphite element 30 as shown in FIG. 2. In another embodiment, the frequency generating device 30 can be constructed from any other suitable carbon-based material, ceramic, or a composite stone material. The frequency generating device can be curvy or winding in shape so as to provide close proximity to the at least the majority of the FIR heat emitting elements 14. The frequency generating device 30 can receive electrical power from its connection to a power transfer junction box 26 that can be connected to a power cord 28. The power cord 28 can be connected to a power source 32 such as, for example, an outlet connected to an electrical system or electric grid. The frequency generating device 30 can installed inside the pouch 16 so as to be hidden from view when the front and rear sheets of material 12a and 12b are stitched together.

As the polygraphite element 30 resonates while connected to a power source, the resonance produces far infrared radiation as well as other electromagnetic radiation from the infrared spectrum (e.g., near infrared radiation). The far infrared radiation produced by the resonating polygraphite element 30 is emitted as radiation through the front sheet of material 12a of the pad 12 to penetrate the user's skin. In addition, the far infrared radiation emitted by the polygraphite element 30 can cause the FIR heat emitting elements 14 to resonate and to emit additional far infrared radiation. The far infrared radiation produced by the FIR heat emitting elements 14 can penetrate the user's skin via conduction, or direct contact between the user's skin and FIR heat emitting elements.

The device 10 can further include a remote control 34 that may feature controls such as, for example, temperature control, a timer (e.g., a digital timer), a sleep timer with automatic power shut-off, and a power switch to turn the device off and on. The temperature control may include displays connected to sensors positioned within the pad to monitor and alert the user as the device 10 reaches the desired temperature (e.g., a temperature selected by the user). The remote controller can include a display screen. The display screen of the remote control 34 may include a backlight (e.g., a xenon backlight). The remote control 34 may be connected to the device 10 via a wired or a wireless connection. The remote control 34 may be either digital or analog.

A mesh material 38 can be attached to an exterior surface 24 of the front sheet of material 12a. The mesh material 38 can be sewn or stitched to attach it to the front sheet of material 12a. In an exemplary embodiment, the mesh material 38 may be stitched to the exterior surface 24 of the front sheet of material 12a to form a plurality of individual cells 22. Each of the plurality of cells 22 may contain an individual FIR heat emitting element 14. The cells 22 may be stitched or sewn completely closed to prevent the plurality of FIR heat emitting elements 14 from falling out or otherwise moving in position or becoming dislodged. In another embodiment, each cell may contain more than one FIR heat emitting element 14. The mesh material 38 can include a plurality of apertures to permit unblocked emission of far infrared heat from the FIR heat emitting elements 14 and radiation and conduction of that far infrared heat to the user's body part that is covered by the pad 12 of the device 10. In an alternate embodiment, a solid sheet of material may be attached to the front sheet of material 12a rather than mesh material.

In an exemplary embodiment, the plurality of FIR heat emitting elements 14 can be arranged and fixed in a regular pattern in the cells 22 of the mesh material 38. The FIR heat emitting elements may also be arranged and fixed in an irregular pattern in the cells 22 of the material 38. In another embodiment, the plurality of FIR heat emitting elements 14 can be fixed in a regular or irregular pattern within the pouch 16 of the pad 12. The FIR heat emitting elements 14 can be sewn into the front sheet of material 12a so that each element is fixed in position. The FIR heat emitting elements 14 may also be connected to the mesh material 38 or to the exterior surface 24 of the front sheet of material 12a by other connecting means such as, for example, snap fasteners, clips, adhesives, plastic ties, sewing or stitching using thread or yarn, or any other suitable connecting means. The connecting means used to attach the FIR heat emitting elements 14 to the pad 12 are heat resistant and fire resistant as are the other materials used in the construction of the device 10. The materials used in the construction of the device 10 can also be sealed for water resistance.

In another embodiment, the front sheet of material 12a of the pad 12 can include a second pouch formed from an additional sheet of material attached to either the exterior surface 24 or interior surface 18 of the front sheet of material. The plurality of FIR heat emitting elements 14 can be attached to either the exterior surface 24 or to the interior surface 18 of the front sheet of material 12a. In another embodiment, the FIR heat emitting elements 14 can be attached to both the interior and the exterior surfaces 18, 24 of the front sheet of material 12a of the pad 12.

In one alternate embodiment, the rear sheet of material 12b of the pad 12 can include a charging inlet for connection to a power source. Where the power source is an electrical system, the device 10 may be connectable to the power source via an electrical cord that can be plugged into an outlet. The charging inlet may be enclosed within a pocket that can be hidden by closing a zipper. In another embodiment, the rear sheet of material may include a pocket that can contain a power source that is a battery.

In one embodiment, the front sheet of material 12a can be two-ply in that it is constructed from two attached sheets of material that may enclose a pouch. Similarly, the rear sheet of material 12b can be two-ply in that it may also be constructed from two attached sheets of material that may enclose a pouch. In another embodiment, the front sheet of material and the rear sheet of material may each be two-ply and when sewn together may enclose a central pouch.

The pad 12 (and the front and rear sheets of material 12a and 12b) may be constructed from any suitable material, e.g., a synthetic or natural material such as vinyl, nylon, polyester, leather, suede, or cotton. The pad 12 can be flexible and may be elastic. The pad 12 may also be constructed from a material that is antimicrobial and antifungal. In another embodiment, the pad 12 may be treated with a composition that imparts antimicrobial and antifungal characteristics to the pad. The pad 12 can be sized to fit over a body part of a human user such as an adult or child. In another embodiment, the pad can be sized and shaped to fit over or around a body part of an animal.

The FIR heat emitting elements 14 can be a stone or mineral material. For example, the FIR heating elements can be jade, germanium, charcoal, rock, porcelain, ceramic, or tourmanium ceramic. The FIR heat emitting elements 14 may be constructed in the shape of discs (circular, oval, or oblong), tiles (square, triangular, rectangular, or rhomboidal), or cubes or the FIR heat emitting elements may be cylindrical, conical, polygonal, or prismoidal in shape. The surface of each FIR heat emitting element can be smooth, convex, concave, or may include a regular or irregular surface texture. The FIR heat emitting elements 14 included with each device 10 may be of the same material, shape, and texture or may vary in material, shape, and/or texture. The FIR heat emitting elements 14 can be arranged in a regular or geometrical pattern or in an irregular pattern. In an exemplary embodiment, the FIR heat emitting elements 14 are jade discs.

In another embodiment, the FIR heating elements 14 can be a man-made, artificial heat conducting or emitting material that may be similar in consistency to stone.

The invention 10 may feature a storage bag or a carrying case. In one embodiment, the invention may also feature an elastic band for securing the pad 12 on, around, or to a chair or other type of seat.

The pad 12 of the FIR heat emitting device 10 can conveniently be laid or worn over or wrapped around a body part of the user to relieve pain such as, for example, lower back pain. The below described preferred embodiments illustrate adaptation of this device and methods for using the device. Nonetheless, from the description of these embodiments, other aspects of the invention can be made and/or practiced based on the description provided below.

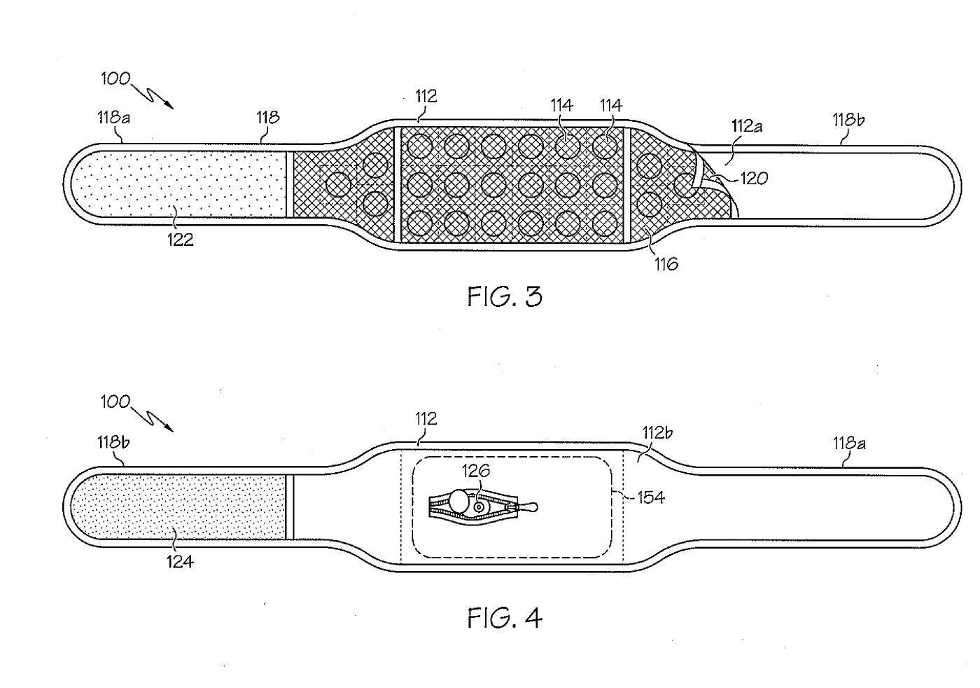

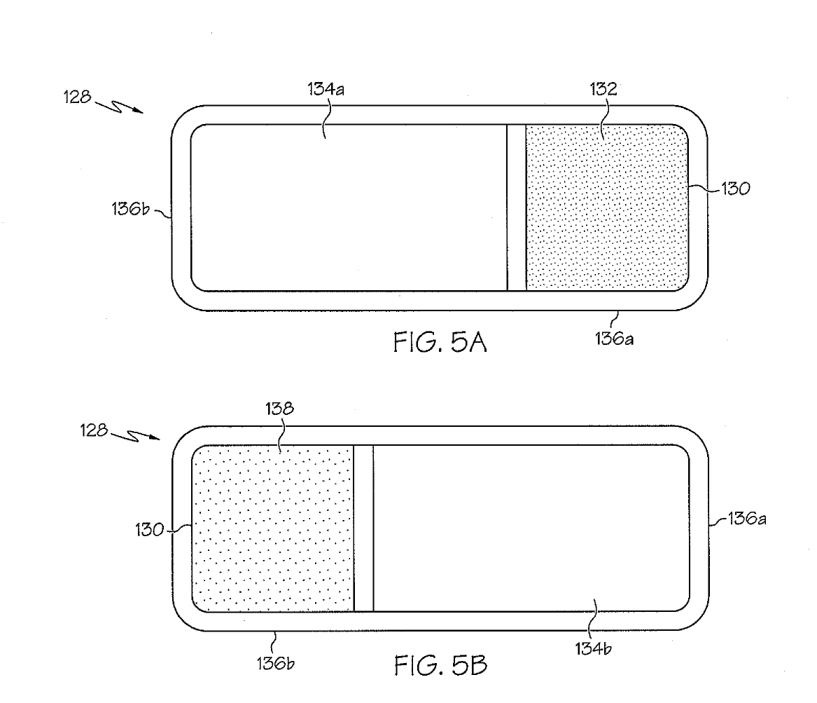

Referring now to FIGS. 3 and 4, an exemplary belt embodiment 100 of the invention is shown. The belt 100 can be used to secure the device 100 around a body part of user such as, for example, a waist, an arm, a leg, a neck, a hand, a foot, a torso, or a back of the user. In an exemplary embodiment, the belt 100 is secured around the waist of a user. The belt 100 can include a pad 112, a plurality of far infrared (FIR) heat emitting elements 114, a device 116 for holding the FIR heat emitting elements 114 in place, and a fastener 118 that forms a belt for securing the pad 112 to a person. The pad 112 can be constructed in a manner and from materials similar to the pad 12 described above and can feature a front sheet of material 112a and a rear sheet of material 112b. The pad 112 may also be constructed from materials that are similar or identical to the materials described above for the pad 12 described above.

The device 116 can be similar to or the same as the mesh material 38 or other materials described herein and used to retain and fix the position of the plurality of FIR heating emitting elements 14 in the pad 12. The device 116 can include the front sheet of material 112a and a mesh material 120 attached thereto as shown in FIG. 3. The mesh material 120 can include apertures that allow the FIR heat emitting elements 114 (e.g., jade discs) to be seen and to allow far infrared heat emitted by the elements 114 to pass through to be absorbed by the user's skin.

The fastener 118 can include first and second straps 118a and 118b integrally attached to and forming a part of the pad 112. One of the straps (e.g., first strap 118a) may include hook fasteners 122 and the other strap (e.g., second strap 118b) may include loop fasteners 124 for connecting to the hook fasteners 122. In one embodiment, to accommodate most adults (e.g., having a waist size of about 28-38 inches), the belt can be approximately 46 inches long by 6 inches wide by 2 inches thick. In other embodiments, the belt can be about 28 to about 52 inches long, about 4 inches to about 12 inches wide, and about 0.5 inches to about 3 inches thick.

In an exemplary embodiment, the belt 100 can include a charging inlet 126 to which a power cord may be connected. In one embodiment, the power cord may be connected to the charging inlet 126 to charge a battery. The battery can serve as the belt's power source. In another embodiment, the power cord may be connected to the charging inlet 126 to provide a direct connection to a power source such as an electrical outlet of an electrical system or grid. The charging inlet 126 may be positioned on the rear sheet of material 112b and may be hidden within a pocket. The pocket may feature a zipper, buttons, snap fasteners, or other fastening means for closing the pocket in order to conceal the charging inlet 126.

The belt 100 can further include a remote control that may include features such as, for example, temperature control, a timer, and a power switch to turn the device off and on. The remote control may be connected to the belt 100 via a wired or a wireless connection.

The pad 112 and plurality of FIR heat emitting elements 114 can be constructed from the same materials and in the same or a similar manner as described above for the construction of the pad 12 and the plurality of FIR heat emitting elements 14. The plurality of FIR heat emitting elements 114 may be held in position and attached to the belt 100 by any suitable method such as sewing into individual pockets or cells, a mechanical or adhesive film fastener, or a hook-and-loop type fastener.

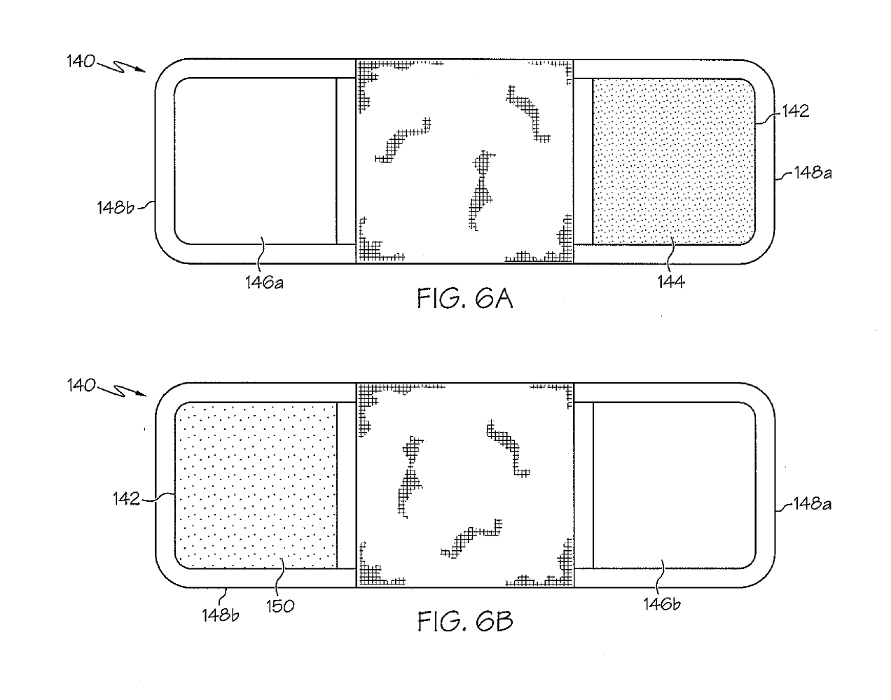

Referring now to FIGS. 5A and 5B, the belt 100 can include a short belt extension 128. The short belt extension 128 can be used to secure the belt 100 around the waist of a user who has a large waist size, e.g., a waist size of 36 to 48 inches. The short belt extension 128 may include fasteners 130 such as, for example, hook fasteners or loop fasteners, to connect to the fasteners 122 or 124 of one or both of the straps 118a and 118b. The short belt extension 128 can include hook fasteners 132 on a first side 134a at a first end 136a of the short belt extension. The short belt extension 128 can include loop fasteners 138 on a second side 134b at a second end 136b of the short belt extension. Thus, the hook fasteners 132 of the short belt extension 128 can be located on a reverse side and at an opposite end of the short belt extension from the position of the loop fasteners 138. In one embodiment, the short belt extension 128 may be constructed at least in part from an elastic material.

The hook fasteners 132 and the loop fasteners 138 of the short belt extension 128 can be connected to the corresponding hook fasteners 122 or loop fasteners 124 of the belt 100 to extend the length of the belt. Hook fasteners are used to connect to loop fasteners. For example, the hook fasteners 132 of the short belt extension 128 can be connected to the loop fasteners 124 of the belt's second strap 118b, and the loop fasteners 138 of the short belt extension 128 can connected to the hook fasteners 122 of the belt's first strap 118a.

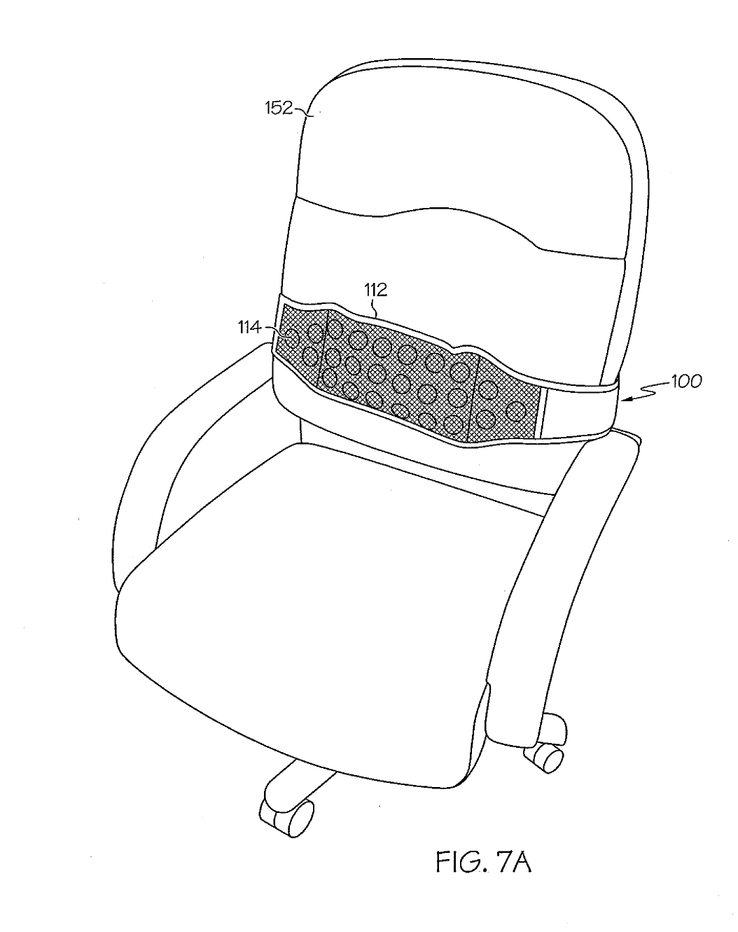

Referring now to FIGS. 6A and 6B, the belt 100 may also include a long belt extension 140 having features similar to those of the short belt extension 128. The long belt extension 140 can be used to secure the belt 100 around the waist of a user having a waist size of greater than 48 inches. The long belt extension 140 may include fasteners 142 such as, for example, hook fasteners or loop fasteners, to connect to the fasteners 122 or 124 of one or both of the straps 118a and 118b. The long belt extension 140 can include hook fasteners 144 on a first side 146a at a first end 148a of the long belt extension. The long belt extension 140 can include loop fasteners 150 on a second side 146b at a second end 148b of the long belt extension. Thus, the hook fasteners 144 of the long belt extension 140 can be located on a reverse side and at an opposite end of the long belt extension from the position of the loop fasteners 150. In one embodiment, the long belt extension 140 may be constructed at least in part from an elastic material.

The fasteners 130 of the short belt extension 128 may be connected to corresponding fasteners 142 of the long belt extension 140 to extend the length of the belt 100 to an even greater degree.

The hook fasteners 144 and the loop fasteners 150 of the long belt extension 140 can be connected to the corresponding hook fasteners 122 or loop fasteners 124 of the belt 100 to extend the length of the belt. Hook fasteners are used to connect to loop fasteners. For example, the hook fasteners 144 of the long belt extension 140 can be connected to the loop fasteners 124 of the belt's second strap 118b, and the loop fasteners 150 of the long belt extension 140 can connected to the hook fasteners 122 of the belt's first strap 118a.

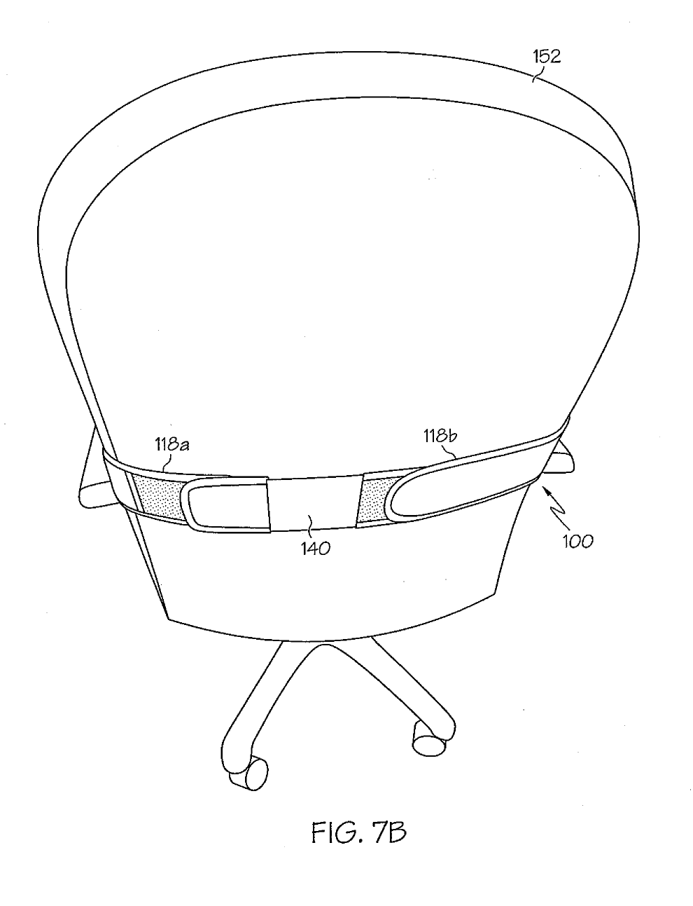

Referring now to FIGS. 7A and 7B, the long belt extension 140 might also be used to attach the belt around a chair 152. The belt 100 can oriented with the front sheet of material configured so that the FIR heat emitting elements 114 would be against the lower back of a user sitting in the chair. In another embodiment, the short belt extension 128 might also be used to attach the belt 100 around a chair 152. In still another embodiment, both the short belt extension 128 and the long belt extension 140 may be connected to the belt 100 and then to one another in order to attach the belt around a chair. The chair 152 can be a seat, a recliner, an office chair, a dining room chair, a straight-backed chair, or any other suitable chair around which the belt 100 may be attached.

Far infrared heat (having a wavelength equal to an including 5.6 to 1,000 micrometers (μm)) can be delivered to the user via the plurality of FIR heat emitting elements 14, 114. In the embodiment shown in FIG. 3, twenty-four circular convex jade discs each 1.5 inches in diameter and 0.3 ounces in weight are arranged in a regular pattern on the exterior surface of the front sheet of material (i.e., the side that abuts against a user when the belt 100 is worn). In other embodiments, a different number of FIR heat emitting elements may be attached to the pad 12 or to the belt 100 (e.g., 6, 7, 8, 9, 10, 12, 15, 20, 22, 25, 29, 30, 35, 40, 50, or 60 FIR heat emitting elements). The number and shape of the FIR heat emitting elements can be selected so that an appropriate amount of heat can be delivered to the user without the elements making the pad uncomfortable for the user to wear due to stiffness and inflexibility.

In other embodiments, the composition, number, shape, and size of the FIR heat emitting elements can be varied. For example, although jade is preferred because of its ability to deliver far infrared heat in a manner preferred by users, other materials such as germanium, charcoal, composite stone, ceramic, and tourmanium ceramic may also be used. Jade's emission wavelength is equal to and includes 4 to 14 μm, which matches so closely to the human body's radiant energy that close to 93% of the infrared waves that reaches the user's skin is absorbed and is able to penetrate up to about 3 inches into tissue and permeate thoroughly the user's skin.

In one embodiment, the FIR heat emitting device 10 or belt 100 can further include a bladder 154, as shown in FIG. 3, in which a heat storing liquid is contained. To store heat, the pad can include a liquid containing bladder in heat conducting communication with the heating device, which can be covered by the pad material and which is electrically connected to the charging inlet. The heating device can be the polygraphite element 30 described above. The liquid contained in the bladder can be any liquid suitable for storing heat. Preferably, the liquid would be an aqueous gel-like viscous solution. The liquid may contain an antimicrobial agent. In one embodiment, the liquid may also contain an anti-freezing agent. The liquid provides the pad with flexibility and shape-conforming properties such that the pad 12 of the device 10 or the pad 112 of the belt 100 can conform to the shape of the user's back so that the pad or belt is comfortable to the user and more efficient at conducting heat. The bladder 154 is preferably entirely covered by the material from which the pad 12 or 112 is constructed and may be covered in an insulating material such as vinyl to minimize heat loss from the liquid.

In both embodiments of the device 10 and 100, the FIR heat emitting elements can be positioned in heat conducting communication with the bladder 154 such that heat stored in the liquid contained therein is gradually transferred to the elements and thereby to a body part of the user wearing the pad 12 or belt 100. The liquid contained in the bladder can be heated by attaching the electrical power cord to the charging inlet and an electrical power source such as an 110V electrical outlet. The current flows from the electrical outlet into the heating device via the electrical power cord and the charging inlet. The heating device transforms the electrical current into heat energy which is delivered to the liquid in the bladder. When the device or belt is fully charged, the liquid contained within the bladder can reach a temperature of about 110 degrees Fahrenheit (° F.). The pad 12 or 112 may also contain a battery which is charged from the 110V electrical outlet via the electrical power cord and the charging inlet. The battery can then gradually discharge its power to heat the liquid. The volume of liquid contained in the bladder can be any suitable for using the pad device or belt to deliver heat to a body part, e.g., the lower back, of the user. In some embodiments, the volume of the liquid in the belt, which may be about 8-10 ounces, is sufficient to maintain the FIR heat emitting elements at a temperature of at least 120° F. for at least about 30 minutes (e.g., at least 30 minutes, 1, 2, 3, 4, 5, 6, 7, 8, 9, or 10 hours). In an exemplary embodiment, the components of the device 10 and belt 100 are selected such that a 15-minute electrical charge can provide enough heat to maintain the FIR heat emitting elements at a temperature of at least 105° F. for at least about 5 hours. To prevent burning, the pad 12 and 112 may also include a thermostat that prevents the pad 12 and 112 from exceeding a temperature of about 140° F.

In an alternate embodiment, the FIR heat emitting device may be incorporated beneath an exterior surface of a piece of furniture. The exterior surface beneath which the FIR heat emitting device is incorporated can be a surface with which the user's body would normally come into contact. For example, the FIR heat emitting device can be installed beneath a seat surface of a chair, recliner, or sofa or beneath a top surface of a mattress.

In another embodiment, the pad may be incorporated on top of the exterior surface of the furniture piece so as to be capable of direct contact with the body of the user.

### Methods of Use

The far infrared heat emitting device of the invention is generally intended to be used to provide far infrared heat to one or more body parts of a user such as, for example, the lower back, arm, leg, or neck of a person, to help relieve pain caused by inflammation or muscle soreness. To use the device, a human user places the pad (or pad of the belt) around or over a selected body part in a position where the plurality of far infrared heat emitting elements are positioned at the site of the user's pain (e.g., the lower back). The pad of the device is left in that position until the pain is relieved, e.g., about 0.2, 0.5, 1, 2, 3, 4, 5, 6, 7, 8 or more hours. Once the pain has diminished, the pad (or belt) may be removed by the user.

In another embodiment of the method, the user can be an animal (e.g., a pet) and a person can place the pad (or belt) over or around and in contact with a body part of the animal.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A therapeutic device for positioning over or around a body part of a user, the device comprising:
a pad comprising a front surface and a rear surface;
a plurality of far infrared heat emitting elements affixed to the pad;
a heating device; and
a power source.

2. The device of claim 1, further comprising a bladder containing a heat storing liquid.

3. The device of claim 1, wherein the pad comprises a fastener to form a belt for securing the pad around a body part of the user.

4. The device of claim 3, wherein the belt is integrated with the pad so that the belt is formed as a single piece.

5. The device of claim 3, wherein the belt comprises a short extension for securing the belt around a waist of size 36-48 inches.

6. The device of claim 3, wherein the belt comprises a long extension for securing the belt around a waist of size 48 inches or greater.

7. The device of claim 3, wherein the device is sized so as to be capable of attaching around a chair.

8. The device of claim 1, wherein the power source comprises a power cord for connection to an outlet of an electrical system.

9. The device of claim 1, wherein the power source comprises a battery.

10. The device of claim 1, wherein the plurality of far infrared heat emitting elements comprise a stone or a mineral.

11. The device of claim 10, wherein the plurality of far infrared heat emitting elements comprise jade.

12. The device of claim 1, wherein the front surface of the pad comprises a pouch, wherein the pouch comprises a lining comprising front and rear interior surfaces.

13. The device of claim 12, wherein the pad comprises individual cells sewn into a lining of the pouch.

14. The device of claim 13, wherein each of the plurality of far infrared heat emitting elements is positioned within one of the individual cells sewn into the lining of the pouch.

15. A far infrared heat emitting device comprising a belt, the belt comprising:
a plurality of far infrared heat emitting elements;
a heating device;
a charging inlet;
a fastener for securing the belt to a user; and
a power source.

16. The device of claim 15, wherein the belt is sized to fit around the waist of the user.

17. A method of administering far infrared heat to a body part of a user, the method comprising the step of:
(a) placing a far infrared heat emitting device in contact with a body part of a user, the device comprising a pad comprising a plurality of far infrared heat emitting elements, a heating device, and a power source.

18. The method of claim 17, further comprising the step of:
(b) leaving the pad of the device in position in contact with the body part of the user for a predetermined amount of time.

19. The method of claim 17, further comprising the step of:
(c) leaving the pad of the device in position in contact with the body part of the user until the user's pain is diminished.

20. The method of claim 17, further comprising the steps of:
(d) equipping the pad with a fastener to form a belt; and
(e) attaching the belt around the body part of the user.
